(19) United States
(12) Patent Application Publication
(10) Pub. No.: US 2006/0073205 A1
(43) Pub. Date: Apr. 6, 2006

(54) Controlled release granules comprising porous silica core

(76) Inventors: Kotoe Ohta, Hyogo-ken (JP); Masayoshi Fuji, Gifu-ken (JP); Takashi Takei, Kanagawa-ken (JP); Masatoshi Chikazawa, Tokyo (JP); Kenzo Toyoshima, Nara-ken (JP)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 10/958,048

(22) Filed: Oct. 4, 2004

(51) Int. Cl. A61K 9/24

(52) U.S. Cl. 424/471

(57) ABSTRACT

The present invention relates to controlled release granules for medical use comprising a drug loaded porous silica particle, and at least one layer of a controlled release coating material, characterized in that the drug loaded porous silica core is prepared by immersing dry porous silica particles with a solution, suspension or emulsion comprising at least one pharmacologically active drug and the resulting wet drug loaded porous silica core is subsequently dried again. Furthermore, the invention concerns a method for preparing the controlled release composition.

### FIELD OF THE INVENTION

The present invention relates to controlled release granules for medical use comprising a drug loaded porous silica, and at least one layer of a controlled release coating material, characterized in that the drug loaded porous silica core is prepared by immersing dry porous silica particles with a solution, suspension or emulsion comprising at least one pharmacologically active drug and the resulting wet drug loaded porous silica core is subsequently dried again. Furthermore, the invention concerns a method for preparing the controlled release composition.

The beneficial efficacy of the compositions and methods according to the invention are based on the simple method of manufacture, the high possible drug load of each silica particle and the high variety of release profiles which can be achieved by variation of the porous silica used and the parameters and composition of the controlled release layer.

### BACKGROUND OF THE INVENTION

As is known in the prior art, it is desirable in the treatment of many diseases to provide the pharmacologically active compound in a controlled release form. This may be due to the necessity to administer the pharmacologically active compound as close as possible to the colon or it may be necessary to eliminate the risk for acidic influence on the compound by the gastric juice, or to prevent from irradiation of the ventricular mucous membranes, or to obtain a therapeutically effect in the lower part of the gastroinestinal tract. A further problem is to obtain a steady, for example, linear release of the pharmacologically active compound in order to give a steady blood plasma level of said compound, without an initial release peak, which may cause side-effects due to too high concentrations in the body.

Porous silica has been widely investigated for controlled release of biologically active substances (see for example Ahola, M. et al., 2000, Int. J. Pharm. 195, 219-227; Bottcher, H. et al., 1998, J. Sol-Gel Sci. Technol. 13, 277-281; Ahola, M. et al., 2001, Biomaterials. 22, 2163-2170; Nicoll, S. B. et al., 1997, Biomaterials 18, 853-859). For that the drug is normally incorporated into the porous silica matrixes during polycondensation of organic silicate, like tetraethyl ortho silicate.

In principle there are two approaches making such sol-gel products. The first method involves gelation of a dispersion of colloidal particles from a drug containing solution; method two employs hydrolysis and polycondensation of organic silicates in drug containing solution followed by supercritical drying of the gels or by aging and drying under ambient atmospheres.

Dependent on the conditions of the polymerization process like pH-value, temperature, organic silicate, additives etc., the release rate of the drug from the porous silica particle is strongly influenced. Thus, the production of such formulations with a reproducible release pattern is very complicated. Furthermore not all drugs can be used in this methods because they will be decomposed under the conditions used for particle production.

The other possibility to prepare pharmaceutical formulations with controlled release patterns is to coat a particle like a non pareille seed with a drug and with a layer of a controlled release material. But such coating films are often ruptured if the coated particles are compressed into tablets, which results in a loss controlled release properties. The rupture of the coating film results from the deformation of the core by the compaction force. This may be prevented by the use of the porous silica cores because they are very rigid in comparison to conventional materials like microcrystalline cellulose or sucrose crystals.

Because of the difficulties with regard to the preparation of sol-gel products and the advantages with respect to the rigidity of the porous silica particles, other possibilities to produce controlled release formulations with a silica gel core have been elaborated.

In WO 01/15751 a pharmaceutical formulation is disclosed comprising a silica gel core and at least two coating layers wherein the drug is incorporated in at least one of the coating layers.

In U.S. Pat. No. 4,925,674 pharmaceutically active microencapsulated granules are disclosed. These granules comprise an inert core (e.g. silica gel) coated with a dispersion comprising a binder and the drug. The granules are preferably enwrapped with a taste mask coating.

However, this formulations have been prepared by coating the silica gel core with a layer comprising the drug. Because the coating process is relatively difficult to carry out, there is a need for methods for the preparation of pharmaceutical formulations with high drug load taking the advantage of the rigidity of porous silica and avoiding the disadvantage of an additional coating step.

### OBJECTIVE OF THE INVENTION

Thus, according to one aspect, this invention concerns a controlled release formulation for medical use in a subject, said formulation comprising
a) a core material consisting of a porous silica particle in which the pharmacological active compound is absorbed,
b) and at least one layer of a controlled release coating material,
   characterized in that the porous silica core comprising the pharmacological active compound is prepared by immersing dry porous silica particles with a solution, emulsion or suspension comprising at least one biological active agent and subsequently drying the resulting drug loaded porous silica core again.

According to a further aspect, the present invention concerns a method for producing a controlled release formulation for medical use in a subject comprising the steps
a) Immersing dry porous silica particles with a solution, suspension or emulsion comprising the pharmacologically active compound in a way that the pharmacologically active compound is absorbed by the porous silica granule,
b) drying the resulting wet porous silica cores so that the solvent is evaporated,
c) coating the resulting drug loaded core with at least one coating material able to control release of the pharmacologically active compound.

A further aspect relates to pharmaceutical dosage forms like sachets, capsules or tablets comprising the granules of the present invention.

### SHORT DESCRIPTION OF THE FIGURES

FIG. 1: Schematic diagram of the production of the controlled release granules according to the present invention.

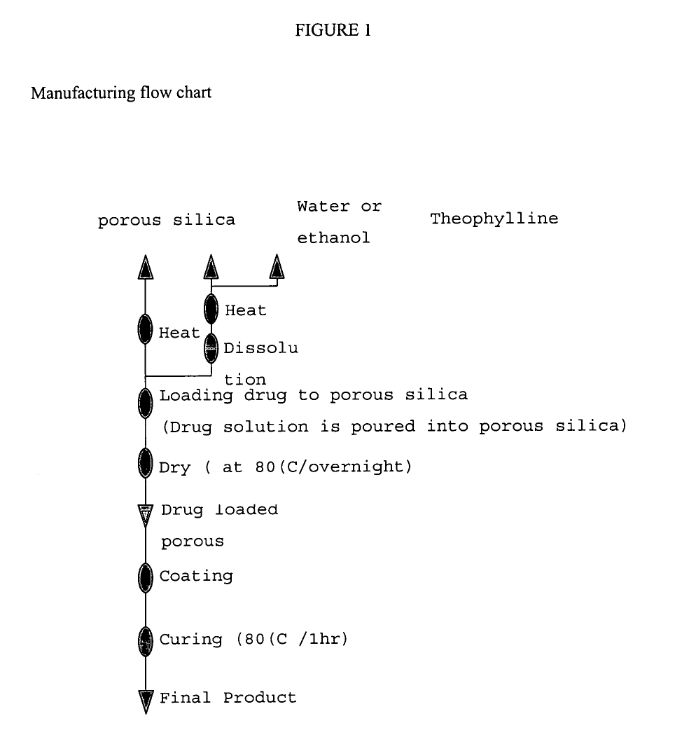

FIG. 2: Graph showing the drug release profile of different formulations uncoated or coated with a controlled release layer according to the present invention.

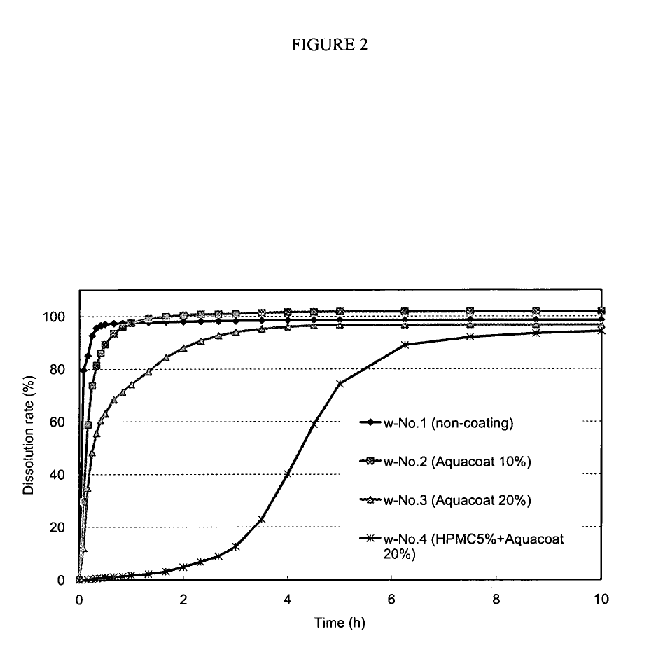

### DESCRIPTION OF THE INVENTION

The present invention provides controlled release granules for medical use comprising 1) a porous silica core in which a pharmacologically active compound is absorbed and 2) at least one layer of a controlled release coating material. The invention provides furthermore methods for the manufacture of such granules.

It now has surprisingly been found that dried porous silica particles have advantageous properties with respect to hardness, friability, the ability to absorb solutions of pharmacologically active compounds and with respect to the properties of the dried drug loaded porous silica cores to release the absorbed drugs almost immediately. Therefore, dried porous silica can excellently be used for the manufacture of controlled release granules.

Additionally to the rigidity, the porous silica particles have the advantage that they can absorb great quantities of a drug (up to about 500 mg/g of porous silica), if they are used according to the present invention. The quantity of absorbed drug is much higher as it can be achieved, for example, by spraying in fluid bed.

Principally, the present invention is not limited to a certain type of porous silica particles as long as the release profile of the controlled release formulation is not (or almost not) influenced. Porous silica particles suitable according to the present invention are rigid and large enough for coating process and independent of manufacturing process. Porous silica obtained either from liquid phase or vapour phase can be applied. Porous silica suitable according to the present invention can for example be regular, intermediate, or low density porous silica.

Regular density porous silica is made in an acid medium, which gives high (e.g. 750 m²/g) surface area, small ultimate particles having average pore diameters of 2.2-2.6 nm, and a pore volume of 0.37-0.40 ml/g. The gel exhibits a high selectivity for polar molecules and has a large percentage of small pores.

Intermediate density porous silica consists of larger ultimate particles having a lower (300-350 m²/g) surface area, larger (0.9-1.1 ml/g) pore volumes, and larger (12-16 nm) average pore diameters. Because of the large pore size, intermediate density porous silica has a high capacity for water adsorption at high humidities. It is often used as a fine powder because aggregate (or secondary) particle size and porosity can be controlled.

Low density porous silica (such as an aerogel) has lower (100-200 m²/g) surface areas, larger (18-22 nm) average pore diameters, and larger (1.4-2.0 ml/g) pore volumes. It is usually prepared as a very fine powder of extremely low density. Shrinkage of the gel during drying is minimized.

By “drug” or “pharmacologically active compound” it shall be understood an agent causing a valuable effect in vivo, such as a bioactive effect, a therapeutic effect, or the like. A pharmacologically active compound can be any organic, inorganic or living agent that is biologically active. It can be a protein, a polypeptide, a polysaccharide (e.g. heparin), an oligosaccharide, a mono-or disaccharide, an organic compound, an organometallic compound or an inorganic compound containing any element. It can be a living or dead cell, bacterium, a virus or a part thereof. It can be a biologically active molecule such as a hormone, a growth factor, a growth factor producing virus, a growth factor inhibitor, a growth factor receptor, an integrin blocker (e.g. a IIa/IIIb inhibitor) or a complete or partial functional gene in sense or antisense orientation in a suitable expression vector or in any other expression vector construct for local delivery of therapeutically active agents. Pharmacologically active agents include those especially useful for long-term therapy, such as hormonal treatment, for example contraception and hormone replacement therapy, and for treatment of diseases such as osteoporosis, cancer, epilepsy, Parkinson's disease and pain. The suitable biologically active agents may be, e.g. anti-inflammatory agents, anti-infective (e.g. antibiotics and antiviral agents), analgesics and analgesic combinations, antiasthmatic agents, anticonvulsants, antidepressants, antidiabetic agents, antineoplastics, anticancer agents, antipsychotics, agents used for cardiovascular diseases.

The preparation of the porous silica core loaded with a pharmacologically active compound is carried out by immersing dry porous silica particles as defined above and below with a solution, suspension or emulsion of the pharmacologically active compound in a suitable solvent/liquid. In principle all solvents/liquids which do not destroy the porous silica particles can be used. Suitable solvents/liquids include but are not limited to water, acetone, ethanol, methanol, isopropanol, chloroform, methylene chloride, methyl ethyl ketone, ethyl acetate, carbon tetrachloride, benzene and combinations thereof. If sufficient solubility of the pharmacologically active compound in the solvent is difficult to achieve, the solvent can be heated in order to achieve better solubility. In this case, the porous silica particles have to be heated to a temperature which is higher as the temperature of the heated solvent/liquid prior to the immersion step to prevent precipitation of the drug on the surface of the porous silica particles. As it is important that all of the drug solution is absorbed in the immersion step, the absorption capability of the porous silica particles has to be determined prior to the immersion step. Absorption capability is checked by adding drug free solvent up to the pores of the porous silica is filled and the outer surface of the particle becomes wet.

If a high drug load of the formulation is desired, porous silica particles with high surface area per unit volume may be used. In contrast, if only a low drug load is desirable, porous silica particles having a low surface area per unit volume can be used. However, it is not essential to use a lower surface area for a low drug load. The drug load of the porous silica particles can be also be influenced by the concentration of the drug solution. If a high drug concentration is used for immersion of the porous silica particles, the drug load of the particles is higher as when a lower concentration is used.

The size the size of the uncoated porous silica particles is normally in the range of 10 μm to 5 mm (average diameter), preferably in the range of 100 μm to 2 mm (average diameter), more preferably in the range of 200 μm to 0.5 mm (average diameter). However with regard to the particle size, the performance of the coating machine has to be taken into account After the dry porous silica particles have been immersed with the solution of the pharmacologically active compound, and all of the solution has been soaked up by the particles, they have to be dried again. Drying of the drug loaded porous silica cores can be performed according to conventional processes known to the person skilled in the art. For example, it may be carried out by any suitable method like lyophilisation, tray drying, convection drying, microwave drying, contact drying, drying with infrared radiation. Parameters like drying time, drying temperature and number of drying cycles have to be adapted to the drug and solvent used, residual moisture acceptable etc. Details of the process are known to the person skilled in the art and are described, for example, in Pharmazeutische Technologie, Chapter 13, p. 414-443 (Springer-Verlag, Berlin Heidelberg New York, 1998).

The so prepared dried drug loaded porous silica cores are coated with one ore more controlled release layer(s). With regard to the controlled release coating, any pharmaceutically acceptable material can be used. Furthermore the formulation can comprise seal coatings to separate various functional layers. Also layers for masking taste or odour can be applied. A formulation according to the present invention can be composed, for example, of a drug loaded porous silica core surrounded by a first inner controlled drug release layer (e.g. diffusion-controlled) a second layer (e.g. a seal layer), surrounding said first layer and separating it from a third layer, which might be an enteric-coating layer. Finally a fourth layer, for example a taste-masking layer may be applied.

Further suitable examples of controlled release formulations are, for example, described in:
Sustained Release Medications, Chemical Technology Review No. 177. Ed. J. C. Johnson. Noyes Data Corporation 1980.
Controlled Drug Delivery, Fundamentals and Applications. 2nd Edition. Eds. J. R. Robinson, V. H. L Lee. Marcel Dekker Inc. New York 1987.

Within the meaning of the present invention the expression “controlled release” means any formulation technique wherein release of the active substance from the dosage form is modified to occur at a slower rate than that from an immediate release product, such as a conventional swallow tablet or capsule. The term “controlled release” includes formulations exhibiting a slow release, delayed release, sustained release, pulsed release or comparable release profiles.

Release controlling polymers include hydrogel polymers, hydrophobic polymers and enteric, or pH dependent polymers.

Suitable materials for the formation of hydrogel or swellable and/or gellable polymers may be selected from alkyl celluloses, hydroxyalkylcelluloses, polyvinyl alcohol, polymethacrylates, polymethylmethacrylates, methacrylate/divinylbenzene copolymers, carboxymethylamide, polyoxyalkylene glycols, polyvinyl pyrrolidone and carboxymethyl cellulose. The swellable polymeric material in particular may be selected from crosslinked sodium carboxymethylcellulose, crosslinked hydroxypropylcellulose, polyhydroxypropylmethylcellulose, carboxymethylamide, carboxy-methyl starch, potassium methacrylate/divinylbenzene copolymer, crosslinked polyvinylpyrrolidone and polyvinyl alcohol.

The gellable polymeric material in particular may be selected from methylcellulose carboxymethylcellulose, low-molecular weight hydroxypropylmethylcellulose, low-molecular weight polyvinylalcohols, polyoxyethyleneglycols and non-cross-linked polyvinylpyrrolidone. The swellable and gellable polymeric material in particular may be selected from medium-viscosity hydroxypropylmethylcellulose and medium-viscosity polyvinylalcohols.

Suitable materials for the formation of hydrophobic release controlling polymer coatings include alkyl celluloses, which may be used in the form of latex suspensions such as Surelease® (Colorcon GmbH, Germany) or cellulose acetate phthalate (Aquacoat® CPD; FMC, Germany), and methacrylic acid derivatives, which may be used in the form of latex suspensions such as Eudragit® RS, RL and NE (Röhm Pharma, Germany).

Suitable waxes for release controlling coating include non-ionic beeswax derivatives such as Gelucire® 62/05. 50/02 or 50/13 (Gattefossé Germany, Germany), glyceryl behenate, other fatty acid mono-, di- or trimesters of glycerol such as Precirol® ato 5 (Gattefossé Germany, Germany), microcrystalline wax, hydrogenated castor oil or hydrogenated vegetable oil, long-chain aliphatic alcohols such as stearyl alcohol and carnuba wax.

The insoluble membranes can also contain a permeability improving compound. Such permeability improving compounds are hydroxypropylmethylcellulose, hydroxypropylcellulose, hydroxyethylcellulose, methylcellulose, polyethylene glycole, fatty acids, or polyvinylpyrrolidone.

Suitable materials for the formation of enteric or pH dependent polymer coatings include methacrylic acid derivatives, which may be used in the form of latex suspensions such as Eudragit® L and S (Rohm Pharma, Germany), Aquacoat® CPD, hydroxypropyl methylcellulose phthalate (HPMCP), polyvinyl acetate phthalate, hydroxypropyl methylcellulose acetate succinate, shellac, cellulose acetate trimellitate, carboxymethylcellulose, copolymers of maleic acid and phthalic acid derivatives and mixtures thereof.

Furthermore, partially acid-soluble components may be selected from polymers such as polyvinyl pyrrolidone, hydroxypropyl cellulose, hydroxypropyl methylcellulose, polyethylene glycol, polyvinyl alcohol and monomers therefor such as sugars, salts, or organic acids and mixtures thereof.

In addition to the controlled release coating the granules of the present invention can be coated with a taste mask coating. As such a taste mask coating a mixture of 35% to about 55% by weight ethylcellulose and about 45% to about 65% by weight of polyethylene glycol.

Seal coats, film layers used to separate the various functional layers of the formulation, the drug loaded core from the first functional layer, or to provide a final layer to the outside of the formulation, contain suitable materials for film forming such as alkylcelluloses, which may be used in the form of latex suspensions such as Surelease® (Colorcon GmbH, Germany) or Aquacoat® ECD (FMC Germany, Germany), or Eudragit® L30D-55 and hydroxyalkycelluloses such as hydroxypropylmethyl-cellulose (for example Opadry® (Colorcon GmbH, Germany)).

The drug loaded porous silica granules according to the invention can additionally contain further pharmaceutically tolerable additives, formulation aids such as suspending agents, stabilizers and/or dispersants or plasticizers both in the core and in the coating. Examples of pharmaceutically tolerable additives include polyvinylpyrrolidone, microcrystalline cellulose, silica, magnesium stearate, lactose, cornstarch, talc, titanium dioxide and polyethylene glycol etc.

The plasticizers may function to improve the physical stability of the controlled release coating. A plasticizer is particularly preferred where the polymer has a high glass transition temperature and/or is of a relatively low molecular weight. The plasticizer may be present in any suitable effective amount. The plasticizer may be selected from diethyl phthalate, dibutyl phthalate, dibutyl sebacate, triethyl citrate, triethyl acetyl citrate, triacetin, tributyl citrate, polyethylene glycol, glycerol, or medium chain triglycerides and the like. It will be understood that the plasticizer used may be largely dictated by the polymer used in the coating formulation, and the compatibility of the plasticizer with coating solution or dispersion. It should be noted that acid or water soluble plasticizers can also be used to function as partially acid soluble component.

The coating is then applied, e.g. sprayed on, according to the usual preparative procedures, for example, as described in Remingtons Pharmaceutical Sciences, 18^{th} Edition, Chapter 90, p. 1671-1675 (Mack Publishing Company, 1990). Depending on the desired drug release properties and properties of the drug loaded porous silica cores, the coating may be accomplished at a appropriate predetermined rate and temperature using a coating pan or a fluid bed drier, for example, a top spray system, the Wurster bottom spray coater or the tangential spray coating system.

After the coating process a curing step may be necessary, depending on the coating material used. The curing process is conducted by any suitable method like tray drying, convection drying, microwave drying, contact drying, preferably in the range of 50 to 100 □C.

However, the optimal process parameters are depending on, the drug used, the substances used for coating, the release desired profile etc., and have to be determined by a person skilled in the art using routine experimentation.

The resulting granules are suitably combined to give single dose units. For this purpose they can be enclosed by any desired pharmaceutically suitable envelope. The granules can be in the form of any suitable pharmaceutical dosage form like capsules, tablets, or in the form of sachets.

For a tablet formulation the drug loaded porous silica granules coated with the controlled release layer(s) can be blended with suitable formulation aids like microcrystalline cellulose, lactose, silicon dioxide, and magnesium stearate and subsequently compressed to tablets (granule tablet). Such a tablet disintegrates rapidly, releasing the controlled release coated granules.

The formulations described in the present invention are explained by the following examples. However, it should be understood that the following description is illustrative only and should not be taken in any way as restriction on the generality of the invention specified above.

### EXAMPLES

### Example 1

Theophylline and purified water were used as a pharmacologically active compound and solvent, respectively. Theophylline was added into water up to 0.1 g/mL and heated at 80° C. in order to achieve better solubility. Theophylline solution was then poured into preheated porous silica. All drug solution was absorbed to silica pore in this immersion step. Solution volume poured was 1 mL/g which was lower than predetermined absorption capability of silica. Specific surface area of porous silica used in this example was about 300 m²/g and its pore volume was 1.0 mL/g. The particle size distribution range of the porous silica was about 0.85 mm to 1.7 mm. Drug loaded porous silica was dried overnight at 80° C. by tray drier. In the next step, drug loaded porous silica was coated with HPMC (Hydroxypropylmethylcellulose) and Aquacoat® ECD by conventional fluid bed spray coating machine. Triethyl citrate was incorporated into Aquacoat® ECD as a plasticizer (20 g triethyl citrate per 80 g ethylcellulose). Optionally HPMC was coated as the first layer at 5 wt % (5 g HPMC per 100 g dried drug loaded porous silica particles) followed by the second seal coating at 10 wt % or 20 wt % with Aquacoat® ECD. In the last step, curing of coated drug loaded porous silica was carried out at 80° C. by tray drier for one hour to form the smooth seal coating film.

### Example 2

The dissolution profile was determined using JP XIV dissolution method II (paddle method) at a constant temperature 37° C. The volume of dissolution test was 900 mL and the paddle rotation speed was 50 rpm. The absorbance value of the dissolution sample were measured with a UV spectrometer at the maximum absorbance of theophylline(A₂₇₁). Test fluid was distilled phosphate buffer pH 6.8. This fluid was prepared by dissolving 3.40 g of KH₂PO₄, 3.55 g of Na₂HPO₄ and water to 2000 mL.

1. A controlled release granule formulation for medical use in a subject, said formulation comprising
a) a core material consisting of a porous silica particle in which the pharmacological active compound is absorbed,
b) and at least one layer of a controlled release coating material.

2. A formulation according to claim 1 characterized in that the porous silica core comprising the pharmacological active compound is prepared by immersing dry porous silica particles with a solution, suspension or emulsion comprising at least one biological active compound and subsequently drying the resulting drug loaded porous silica core again.

3. The formulation according to claim 1 wherein the formulation comprises only one controlled release layer.

4. The formulation according to claim 1 wherein the formulation comprises two controlled release layers.

5. The formulation according to claim 1 wherein the formulation comprises more than two controlled release layers.

6. The formulation according to claim 1 wherein the size of uncoated drug loaded porous silica core is about 10 μm to 5 mm average diameter.

7. The formulation according to claim 6 wherein size of the uncoated drug loaded porous silica core is about 100 μm to 2 mm average diameter.

8. The formulation according to claim 6 wherein size of the uncoated drug loaded porous silica core is about 200 μm to 0.5 mm average diameter.

9. A pharmaceutical dosage form, which comprises controlled release granules according to claim 1.

10. A pharmaceutical dosage form according to claim 7, selected from the group consisting of a sachet, a capsule or a tablet.

11. A method for producing a controlled release formulation for medical use in a subject comprising the steps:
a) immersing dry porous silica particles with a solution, suspension or emulsion comprising the pharmacologically active compound in a way that the pharmacologically active compound is absorbed by the porous silica granule,
b) drying the resulting wet porous silica cores so that the solvent is evaporated,
c) coating the resulting drug loaded core with at least one coating layer able to control release of the pharmacologically active compound.

12. The method according to claim 9 wherein the drug loaded wet porous silica cores are dried by tray drying.

13. The method according to claim 9 wherein the porous silica cores of step b are coated with one controlled release layer.

14. The method according to claim 9 wherein the porous silica cores of step b are coated with two controlled release layers.

15. The method according to claim 9 wherein the porous silica cores of step b are coated with three controlled release layers.

16. The method according to claim 9 wherein the size of uncoated drug loaded porous silica core is about 10 μm to 5 mm average diameter.

17. The method according to claim 9 wherein the size of uncoated drug loaded porous silica core is about 100 μm to 2 mm average diameter.

18. The method according to claim 9 wherein the size of uncoated drug loaded porous silica core is about more preferably in the range of 200 μm to 0.5 mm average diameter.
